(19) United States
(12) Patent Application Publication
(10) Pub. No.: US 2009/0092968 A1
(43) Pub. Date: Apr. 9, 2009

(54) SCREENING METHOD FOR MYCOBACTERIA IN METAL REMOVAL FLUID SYSTEMS

(75) Inventors: James Benjamin D'Arcy, Attica, MI (US); Donald John Smolenski, Grosse Pointe, MI (US); James D. Griffin, JR., Rochester, MI (US); David L. Thorpe, Orion, MI (US)

Correspondence Address: General Motors Corporation;c/o REISING, ETHINGTON, BARNES, KISSELLE, P.C., P.O. BOX 4390, TROY, MI, 48099-4390, US

(73) Assignee: GM GLOBAL TECHNOLOGY OPERATIONS, INC., Detroit, MI (US)

(21) Appl. No.: 11/867,478

(22) Filed: Oct. 4, 2007

(51) Int. Cl. C12Q 1/24; C12Q 1/68

(52) U.S. Cl. 435/6; 435/30

(57) ABSTRACT

One embodiment of the invention includes a method for testing a fluid for contamination by a mycobacterial organism. A plurality of individual standardized samples may be acquired from a plurality of predetermined sampling points and pooled into a pooled sample. The pooled sample may be analyzed using a polymerase chain reaction technique and the results compared against a pool concern level which is calculated from an individual concern level that governs each individual standardized sample.

### TECHNICAL FIELD

This invention generally relates to screening methods for detecting various forms of mycobacterial contamination within a fluid.

### BACKGROUND

In the manufacture of a product or component of a product it may be necessary to grind, cut, broach, hone, tap, or otherwise machine a metal substrate with the assistance of a tool. In performing these operations, it is customary to utilize a metal removal fluid (MRF) to improve product quality and life of the tool. For instance, an MRF usually provides some level of lubrication between the metal substrate and the tool, and also serves as a coolant to dissipate heat generated during the metal manufacturing operation. Additionally, MRF's may contain various additives that, among other things, stabilize the fluid at more extreme operating conditions, improve lubricity, provide for more effective cooling, decrease susceptibility to corrosion, avoid excess foam formation, and aid in the removal of fine abrasive metal particles. As a result of their numerous benefits, a wide variety of MRF's are available in the marketplace and include, for example, DA Stuart T-Kool 145, Quaker Quakeral 377.

The attractiveness of these and other MRF's in the manufacturing sector have prompted some industries to develop testing procedures and quality control measures to monitor usage and bath life. One such procedure evaluates MRF's for possible contamination by mycobacteria. As understood by those skilled in the art, mycobacterial organisms can exist in a wide array of water sources and therefore possess the capability to contaminate MRF's that comprise water at any concentration. Furthermore, mycobacteria tend to be fastidious. For example, it is possible that a mycobacterial specimen may take several years to develop in culture.

Additionally, some species exhibit relatively long reproductive cycles that can require several weeks to proceed through a single cycle.

### Summary of Exemplary Embodiments

One embodiment of the invention comprises a method for detecting mycobacterial contamination in a fluid such as a metal removal fluid. A plurality of samples may be acquired and consolidated into a pooled sample for testing. The pooled sample may be analyzed for mycobacterial contamination by a polymerase chain reaction procedure.

Another embodiment of the invention comprises a method for detecting a known fluid constituent in a fluid. A plurality of samples may be acquired and consolidated into a pooled sample for testing. The pooled sample may be analyzed and compared against a concern level different than the concern level that governs each sample that is consolidated into the pooled sample.

Other exemplary embodiments of the disclosure will become apparent from the detailed description. It should be understood that the detailed description and specific examples, while indicating the exemplary embodiments of the disclosure, are intended for illustration purposes only and not intended to limit the scope of the invention.

### BRIEF DESCRIPTION OF THE DRAWINGS

Exemplary embodiments of the invention will become more fully understood from the detailed description and the accompanying drawings. The following is a brief description of the drawings.

FIG. 1 is a block diagram setting forth one embodiment of the invention.

### DETAILED DESCRIPTION OF EXEMPLARY EMBODIMENTS

The description of the following embodiment(s) is merely exemplary in nature and is in no way intended to limit the claimed invention, its application, or its uses.

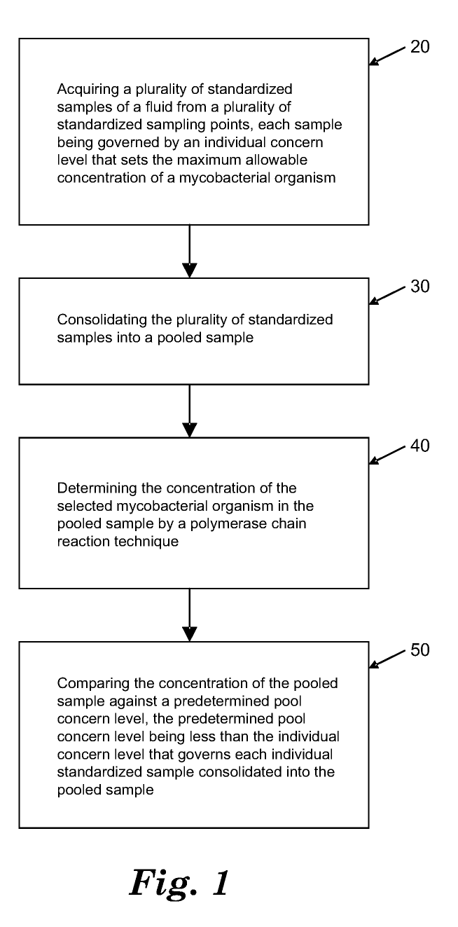

Referring now to FIG. 1, a plurality of individual standardized samples may be obtained from one or more sample points, as shown by numeral 20. The one or more sample points may include any location where a MRF may be collected, such as central distribution or storage system, or an individual tool or machine. Each individual sample may be governed by an individual concern level that reflects the maximum allowable concentration of mycobacteria that may be present in a MRF sample. Individual concern levels are normally determined by those in the industry and may vary depending on factors such as the type of industry or the particular metal manufacturing process for which the MRF is utilized. If the individual sample fails to show a mycobacteria concentration less than the individual concern level, appropriate corrective action may be required to reduce the contamination and restore the source of the MRF to proper operating specifications.

As shown by numeral 30, the plurality of individual standardized samples may be consolidated into a one or more pooled samples prior to testing for mycobacterial contamination. The use of pooled samples reduces the total number of samples that eventually get tested and may lead to a significant reduction in costs associated with mycobacterial assays. The number of individual samples consolidated into a pooled sample depends on the likelihood of finding mycobacterial contamination above the individual concern level in the systems represented by the individual samples. For example, it may be practical to consolidate into a pooled sample a known number of individual samples believed to be free of mycobacteria contamination. Historical data and system specific components or controls may aid in determining the sources of individual samples that are to be pooled.

Next, as shown in numeral 40, a direct measurement of the mycobacterial contamination of a pooled sample may be determined. For example, in one embodiment, the pooled sample may be analyzed using a polymerase chain reaction (PCR) technique. A PCR technique is a relatively quick and very sensitive analytical technique that allows for a target DNA fragment of a DNA structure to be amplified exponentially. In fact, PCR techniques can theoretically detect the presence of a single DNA structure comprising the target DNA fragment in a given sample. In the case of mycobacterial detection, the target DNA fragment is specific to the mycobacteria being tested for and serves to distinguish it against all other organisms that may be present in the sample. Any PCR assay known to those skilled in the art may be used to analyze a pooled sample for the presence of mycobacterial contamination. The basis steps of a general PCR technique are briefly described at a later point.

As alluded to above, a properly designed PCR technique may be an effective and accurate technique for directly determining the concentration of mycobacteria in a pooled sample. However, because the identity of each individual standardized sample is eliminated upon pooling, the individual concern level is no longer an appropriate standard against which the results of the mycobacterial testing should be compared. Instead, referring now to numeral 50, the concentration of a mycobacterial organism in a pooled sample may be compared against a pool concern level, which takes into consideration the individual concern level and the number of samples consolidated into the pooled sample.

In one embodiment, the pool concern level may be calculated by dividing the individual concern level by the number of individual standardized samples consolidated into the pooled sample. Calculating the pool concern level in this manner guarantees that each individual sample consolidated into a pooled sample possessed a mycobacterial concentration lower than the individual concern level if the pooled sample shows a mycobacterial concentration lower than the pool concern level. In other words, it can be mathematically established that no individual standardized sample contained a mycobacterial concentration above the individual concern level if the pooled sample shows a mycobacterial concentration lower than the pooled concern level. This is true even if the entire mycobacteria contamination in the pooled sample resulted from a sole individual sample. Accordingly, the testing of a single pooled sample may verify that numerous individual standardized samples contain a mycobacterial concentration level that is below the individual concern level. The ability to test individual standardized samples in this manner may lead to, among others, significant time and cost savings in the manufacturing sector.

However, no guarantee can be made that each individual standardized sample comprised a mycobacterial concentration below the individual concern level if the pooled sample shows a mycobacterial concentration greater than the pool concern level. In this case, the systems or tools that produced the individual standardized samples may need to be individually analyzed. Furthermore, a pooled sample that shows a mycobacterial contamination greater than the pool concern level does not automatically denote that one or more individual standardized samples are in excess of the individual concern level. Instead, a failing pooled sample simply reveals that it is mathematically possible for a single individual standardized sample to have a mycobacterial concentration above the individual concern level. It is therefore conceivable that a pooled sample that shows a mycobacterial concentration above the pool concern level may, after individual testing, be found to contain a plurality of individual standardized samples that each exhibit mycobacterial concentrations below the individual concern level.

The following examples illustrate possible results that may be obtained when practicing certain embodiments of this invention. In each example, three standardized samples of 10 ml each are consolidated into a pooled sample. The individual concern level for each individual sample is 0.1 g/ml, and thus the pool concern level is 0.033 g/ml. The examples show the mycobacterial contribution each individual sample makes to the pooled sample and how the results are analyzed.

### EXAMPLE 1

This example illustrates a situation in which the pooled sample shows a mycobacterial concentration below the pool concern level. The mycobacterial concentration and the total amount of mycobacteria in each individual standardized sample are listed below.

INDIVIDUAL STANDARDIZED SAMPLES

| Sample # | Myco. Conc. (g/ml) | Total Myco. (g) |
|---|---|---|
| 1 | 0.050 | 0.50 |
| 2 | 0.022 | 0.22 |
| 3 | 0.016 | 0.16 |

A user, not knowing the above individual concentrations, would consolidate the three samples into a pooled sample. A PCR analysis would reveal that the pooled sample contains 0.88 g of mycobacteria. The resulting concentration of the pooled sample is thus 0.029 g/ml, which is below the pool concern level of 0.033 g/ml. The systems or tools that produced the individual samples do not need to be separately tested because the individual standardized samples are guaranteed to be below the individual concern level. For example, even if a single individual sample supplied all the mycobacteria in the pooled sample (0.88 g), the concentration of that individual sample would be 0.088 g/ml, which is still below the individual concern level of 0.10 g/ml.

### EXAMPLE 2

This example illustrates a situation in which each individual standardized sample has a mycobacterial concentration below the individual concern level but the pooled sample shows a concentration above the pool concern level. The mycobacterial concentration and the total amount of mycobacteria in each individual standardized sample are listed below.

INDIVIDUAL STANDARDIZED SAMPLES

| Sample # | Myco. Conc. (g/ml) | Total Myco. (g) |
|---|---|---|
| 1 | 0.050 | 0.50 |
| 2 | 0.022 | 0.22 |
| 3 | 0.070 | 0.70 |

Again, a user not knowing the above individual concentrations would consolidate the three samples into a pooled sample. This time, a PCR analysis would reveal that the pooled sample contains 1.42 g of mycobacteria. The resulting concentration of the pooled sample is thus 0.047 g/ml, which is above the pool concern level of 0.033 g/ml. The systems or tools that produced the individual samples would need to be separately tested because there is a mathematical possibility that one of the individual standardized samples may be above the individual concern level. For example, if a single individual sample supplied all the mycobacteria (1.42 g) to the pooled sample, that individual sample would have a concentration of 0.142 g/ml, which is above the individual concern level of 0.10 g/ml. The fact that the source of the contamination in the pooled sample can not be traced to an individual sample requires separate testing of the sources of the three individual samples. The three sources would be tested against the individual concern level and be found compliant.

### EXAMPLE 3

This example illustrates a situation in which the pooled sample shows a mycobacterial concentration above the pool concern level due to the fact that an individual standardized sample is above the individual concern level. The mycobacterial concentration and the total amount of mycobacteria in each individual standardized sample are listed below.

INDIVIDUAL STANDARDIZED SAMPLES

| Sample # | Myco. Conc. (g/ml) | Total Myco. (g) |
|---|---|---|
| 1 | 0.115 | 1.15 |
| 2 | 0.022 | 0.22 |
| 3 | 0.016 | 0.16 |

Just like the first two examples, a user not knowing the above individual concentrations would consolidate the three samples into a pooled sample. This time, a PCR analysis would reveal that the pooled sample contains 1.53 g of mycobacteria. The resulting concentration of the pooled sample is thus 0.051 g/ml, which is above the pool concern level of 0.033 g/ml. Similar to Example 2, the systems or tools that produced the individual samples would need to be separately tested because there is a mathematical possibility that one of the individual samples may be above the individual concern level. In this example, however, that mathematical possibility is a reality and the source that contains a mycobacterial concentration above the individual concern level would be identified while separately testing the three individual sources.

The above described method may be utilized to pool samples and test for other fluid constituents besides mycobacteria. Examples of fluid constituents that may be detected include, but are not limited to, gram negative bacteria and fungus.

The generalities of a basic PCR technique will now be briefly described. But testing for mycobacteria and other fluid constituents may be conducted by a variety of techniques and is not limited to the following method. First, after initial preparation, a sample is heated in order to denature, or separate, any existing double helix DNA structures into two single DNA strands having complimentary nucleotide base sequences. More specifically, the added heat breaks the hydrogen bonds that hold the double-helix structure together to form a first single DNA strand and a second single DNA strand. The purpose of breaking apart the double helix DNA structures is to allow both the first single DNA strand and the second single DNA strand to serve as templates for replicating the target DNA fragment. A conventional temperature range for this denaturing step is about 94-96° C.

Following the denaturation step, the temperature is lowered so that a first primer can attach to the first single DNA strand and a second primer can attach to the second single DNA strand, a process known as annealing. Primers are short, synthetic single-stranded DNA structures comprising base sequences that selectively attach to portions of the single DNA strands where replication is to begin. Normally, the primers are designed to attach to their respective single DNA strands at a location prior to the target DNA fragment so that a subsequent step will replicate only the fragment. Thus, at the conclusion of annealing step, primers are attached to the first single DNA strand and the second single DNA strand at predetermined locations that will subsequently allow exact copies of the target DNA fragment to be synthesized. Of course, if a DNA structure comprising the target DNA fragment is not present in the sample, the primers will not routinely attach to any DNA sequence and the remaining steps of the PCR process will not be able to proceed. The temperature of the annealing stage may vary depending on the composition of the primers and their corresponding melting points. However, the temperature is usually set at about 5° C. below the melting temperature of the primers, which normally falls between 45° C. and 65° C.

After the annealing step, the temperature is raised to allow an enzyme known as DNA polymerase to synthesize the first single DNA strand comprising the first primer and the second single DNA strand comprising the second primer into exact copies of the original double helix target DNA fragment. This step is routinely referred to as the elongation step. During this step, the DNA polymerase seeks out and attaches to the first and second primers which are already attached to their respective single DNA strands. The DNA polymerase then elongates the primers by moving along the remaining portion of the target fragment and adding complimentary nucleotides to the single DNA strands. The end result of the elongation step is the generation of two indistinguishable copies of the target DNA fragment derived from the presence of a single DNA structure comprising the target DNA fragment. The remaining portion of the DNA structure not comprising the target DNA fragment is not replicated. The temperature of the elongation step depends on the particular DNA polymerase enzyme being used. However, a typical temperature range is usually set around 72° C.

As shown to above, a single PCR cycle comprising a denaturing step, an annealing step, and an elongation step doubles the amount of the target DNA fragment identified by the primers. In practice, the cycle is repeated anywhere from twenty to forty times.

Following the PCR cycles, the product derived from the process may be identified by gel electrophoresis. Gel electrophoresis is a procedure characterized by injecting the derived DNA product from the PCR process into an agarose gel and applying an electric current to the gel. This procedure allows the derived DNA product from the PCR process to be compared against a known standard of the target DNA fragment in order to determine if the target DNA fragment is present in the sample. It is also possible to calculate the exact amount of the original DNA structure comprising the target DNA fragment present in the sample. To make this calculation, one has to determine the total amount of the target DNA fragment generated by the PCR process and the number of PCR cycles performed, and then simply work backwards. Likewise, the concentration of the organism comprising the DNA structure that comprises the target DNA fragment may also be calculated.

While exemplary embodiments of the disclosure have been described above, it will be recognized and understood that various modifications can be made by those of ordinary skill in the art. The appended claims are intended to cover all such modifications which may fall within the spirit and scope of the invention.

What is claimed is:

1. A method comprising:
acquiring a plurality of individual standardized samples of a fluid from a plurality of predetermined sampling points, each individual sample being governed by a predetermined individual concern level that sets the maximum allowable concentration of a selected mycobacterial organism;
consolidating the plurality of individual standardized samples into a pooled sample;
determining the concentration of the selected mycobacterial organism in the pooled sample; and
comparing the concentration of the pooled sample against a predetermined pool concern level, the predetermined pool concern level being less than the predetermined individual concern level that governs each individual sample consolidated into the pool sample.

2. A method as set forth in claim 1 wherein the fluid is a metal removal fluid solution that comprises water.

3. A method as set forth in claim 1 wherein the fluid is a metal removal fluid emulsion that comprises water and an emulsifiable oil.

4. A method as set forth in claim 1 wherein the fluid is a metal removal fluid solution that comprises water and a detergent.

5. A method as set forth in claim 1 wherein determining the concentration of the selected mycobacterial organism comprises analyzing the pooled sample with a polymerase chain reaction technique.

6. A method as set forth in claim 1 wherein the predetermined pool concern level is calculated by dividing the individual concern level by the number of standardized samples consolidated in the pool sample.

7. A method as set forth in claim 1 further comprising:
acknowledging that the concentration of the selected mycobacterial organism in each individual standardized sample is below the predetermined individual concern level if the concentration of the selected mycobacterial organism in the pooled sample is below the predetermined pool concern level.

8. A method comprising:
acquiring a plurality of individual standardized samples of a fluid from a plurality of predetermined sampling points, each individual sample being governed by a predetermined individual concern level that sets the maximum allowable concentration of a selected fluid constituent;
consolidating the plurality of individual standardized samples into a pooled sample;
determining the concentration of the selected fluid constituent in the pooled sample; and
comparing the concentration of the pooled sample against a predetermined pool concern level, the predetermined pool concern level being less than the predetermined individual concern level that governs each individual sample consolidated into the pool sample.

9. A method as set forth in claim 8 wherein the selected fluid constituent is a mycobacterial organism.
